(19) United States
(12) Patent Application Publication
(10) Pub. No.: US 2023/0037952 A1
(43) Pub. Date: Feb. 9, 2023

(54) DETECTION OF KINETOSIS

(71) Applicant: ZF Friedrichshafen AG, Friedrichshafen (DE)

(72) Inventors: Mohamad Alayan, Tettnang (DE); Florian Nicola, Merzig (DE)

(73) Assignee: ZF Friedrichshafen AG, Friedrichshafen (DE)

(21) Appl. No.: 17/760,257

(22) PCT Filed: Feb. 3, 2021
(86) PCT No.: PCT/EP2021/052541

§ 371 Date: Aug. 5, 2022

(30) Foreign Application Priority Data
Feb. 6, 2020 (DE) 10 2020 201 444.2

(51) Int. Cl. A61N 1/36

(52) U.S. Cl. CPC A61N 1/36031; A61N 1/36025; A61N 1/36034; A61H 39/002

(57) ABSTRACT

Treating kenosis may comprise the following steps: measuring the electrodermal activity of a person by means of an EDA sensor; assessing, on the basis of the electrodermal activity measured, whether the person is currently affected by kinetosis; generating electrical pulses at an electrode in contact with the person's skin in order to treat the kinetosis on the basis of the assessment as to whether the person is currently affected by kinetosis. The sensor and the electrode may be integrated in a device that can be worn on the person's body.

### FIELD OF THE INVENTION

The present invention relates to a method for treating kinetosis and a device to be worn on the body of a person.

### SUMMARY OF THE INVENTION

The following is accordingly provided:
a method for detecting kinetosis of a passenger in a vehicle having the following steps: measuring an electrodermal activity of a person by means of an EDA sensor; assessing, on the basis of the measured electrodermal activity, whether the person is negatively affected by kinetosis; generating electrical pulses at an electrode which is in skin contact with the person to treat the kinetosis on the basis of the assessment as to whether the person is negatively affected by kinetosis; wherein the sensor and the electrode are integrated in a device wearable on the body of the person.

Physical reactions such as pallor, dizziness, headaches, nausea, and vomiting, which can be triggered by unaccustomed movements, for example in a means of transportation or in a skyscraper without sufficient vibration absorption, are called kinetosis. Seasickness, airsickness, space sickness, or the land sickness of seagoing people walking on land are known forms of kinetosis. Passive movement in buses, automobiles, trains having tilting technology, aircraft, and roller coasters can also induce kinetosis. It is characteristic that the drivers of the respective vehicles almost never fall ill with kinetosis.

Kinetosis can also occur in driving and flight simulators and adventure cinemas. The first form of kinetosis is referred to as simulator sickness; cases of illness among garners (gaming sickness), in particular when using VR glasses (VR sickness), are relatively new.

Electrodermal activity (EDA; Galvanic Skin Response, GSR, Skin Conductance Response, SCR, or Electrodermal Response, EDR) is a short-term change of the electrical conductive resistance of the skin, caused by the typical increase of the sympathetic tone in the event of emotional-affective reactions. Elevated sweat secretion occurs in this case, corresponding to an increase in the electrodermal activity.

EDA is used as a generic term for tonic or phasic phenomena of the skin. The tonic components correspond to the conductance level (“level”). They change only slowly. The conductance level as such results from the filling of the sweat gland ducts, the moisture penetration of the cornea, and the permeability changes of the sweat gland ducts.

Psychophysiological relationships may be objectified with the aid of measurements of the electrodermal activity, since any physiological excitation, as accompanies emotions or stress, changes the electrodermal activity.

Electrical pulses are moving charges, for example in the form of low stimulation current, which stimulates the human nerves or musculature.

An electrode is an electron conductor which, in cooperation with a counter electrode (anode-cathode), interacts with a medium located between the two electrodes. Electrodes comprise electrical conductors, usually a metal or graphite.

Negatively affected means that a person perceives disadvantageous, painful, or unpleasant symptoms. Treatment is understood as measures which alleviate these symptoms.

The body temperature is the temperature of a human body. The term comprises the temperature of the body interior and the surface temperature of the skin.

A sensor, also referred to as a detector, (measured variable or measurement) transducer, or (measurement) probe, is a technical component which can acquire specific physical or chemical properties or conditions, e.g. temperature, humidity, pressure, velocity, brightness, acceleration, pH, ionic strength, electrochemical potential and/or the material composition of its surroundings qualitatively or quantitatively as a measured variable. These variables are acquired by means of physical or chemical effects and reformed as sensor data into an electrical signal which can be further processed. Vehicle sensors are installed on a vehicle to acquire vehicle surroundings. Sensor data which are acquired by vehicle sensors are sensor data related to the vehicle surroundings.

Vehicles are land, air, or water vehicles. The concept of vehicles also comprises vehicle simulators in the present patent application.

Movements of a vehicle cause stimuli on passengers in the vehicle. In the event of a sustained movement of the vehicle, the stimuli are superimposed and result in a stimulus level.

A device wearable on the human body is an object which does not have to be held by a person, but rather can be worn by the person without active force action thereby. These include, for example, wearables. A wearable is worn during use on the body of the user (for example smartwatch, smart glasses) or is integrated into clothing. Other wearables are, for example, finger clips, earrings, helmets, caps, and the like if they have an electronic function.

Meridians, with respect to “pathways”, are channels in which the life energy (qi) flows. According to these concepts, there are twelve main pathways. Each meridian is associated with a functional circuit (organ system). The acupuncture points are located on the meridians, which are treated using needles in acupuncture and using finger pressure in acupressure. Typical acupuncture/acupressure points are cataloged, i.e. named, located, and associated with a physiological function, in a schematic diagram.

Users can input user inputs into a system by means of a user interface. User interfaces can comprise, for example, speech or text input means, buttons, and cameras for acquiring inputs by gestures.

Energy harvesting refers to obtaining small amounts of electrical energy from sources such as ambient temperature, vibrations, or airflows for mobile devices at low power. Energy harvesting avoids restrictions due to wired power supply or batteries in the case of wireless technologies. Piezoelectric effects, wherein electrical voltages are generated upon force action, for example due to pressure or vibration, thermoelectric/pyroelectric effects, wherein electrical energy is obtained from temperature differences, photovoltaics, electrical energy from the ambient lighting, osmosis, or the like, come into consideration for obtaining energy.

Moreover, macroeffects also come into consideration (mechanical, with or without induction). Some wristwatches are driven by the movement of the wrist. A strong imbalance on an axis is usually automatically brought into rotation by the wearer and the rotational energy is supplied mechanically or electromagnetically to the core function of the watch. Variations having electrical linear generators are used, for example, in flashlights.

The underlying concept of the invention is to study whether a person is negatively affected by symptoms of kinetosis, for example nausea. To assess this, the invention provides for measuring the electrodermal activity of a person and drawing conclusions about kinetosis on the basis of the measured electrodermal activity.

If it is established that a person is negatively affected by kinetosis, an electrical treatment, which is directed to the kinetosis, is initiated. For this purpose, electrical pulses are generated at an electrode which is in skin contact with the person.

The effect of the treatment typically occurs due to a stimulation of the nerve system, the connective tissue, and/or the musculature. However, it is obvious that a therapeutic effect can often also be achieved by the so-called placebo effect, and the concept of treatment in the present patent application also comprises an effect of a placebo effect.

The invention can be used in a variety of ways. Above all, technologies which often induce kinetosis of persons come into consideration for the use of the invention. These include, for example, vehicles for passenger conveyance or other utility vehicles having a vehicle occupant; in particular vehicle occupants who are not themselves occupied with driving a vehicle often suffer kinetosis. Furthermore, the method according to the invention or the device according to the invention can also be used in motion simulators, for example flight simulators or VR (virtual reality) simulators.

For example, it is foreseeable on the filing date of the present patent application that motion simulators will in future also be used to carry out invasive interventions with local anesthetic, since it has already been established in studies that patients require significantly less anesthetic or analgesic when they are distracted during an invasive intervention under local anesthetic by a motion simulator, for example in combination with a computer game. Above all, the side effects of anesthetics or analgesics may be reduced in this way.

Advantageous embodiments and refinements result from the further dependent claims and from the description with reference to the figures of the drawing.

According to a further aspect of the invention, an acupuncture point and/or an acupressure point is stimulated by means of the electrical pulses. Although the effect of acupuncture and/or acupressure or the points thereof is not yet completely understood by modern medicine, the positive effect of acupuncture or acupressure on a variety of ailments can be accepted.

According to a further aspect of the invention, an intensity of the electrical pulses can furthermore be input via a user input of the person into a user interface.

The intensity of the electrical pulses may thus be controlled individually by the person themselves. This is advantageous since the individual sensitivity of a person may thus be taken into consideration. It is thus known, for example, that identical stimuli are perceived by different persons at a differing intensity.

According to a further aspect of the invention, furthermore the skin temperature of the person is measured by means of a thermometer and the negative effect is furthermore assessed on the basis of the skin temperature.

More precise conclusions about the extent of kinetosis may thus be made. This also enables the individual physiology of a person to be taken into consideration.

It is clear that a device to be worn on the body of a person having a sensor for measuring the electrodermal activity of the person using an electrode on a surface of the device, a processing unit for assessing whether the person is negatively affected by kinetosis, and for ascertaining an electrical pulse at the electrode for treating the kinetosis, and having a voltage source for the processing unit and the electrode is advantageous.

According to a further aspect of the invention, the device furthermore has a voltage source which is configured to generate voltage by way of the device itself. The methods of so-called energy harvesting come into consideration for this purpose, for example.

According to a further aspect of the invention, the device is shaped in such a way that the correct application of the device for stimulating a specific acupuncture point is ensured. If, for example, a point in the vicinity of the wrist is to be stimulated, it can be provided that the device is formed as a wristband, which is to be applied to the wrist or which is supported on the wrist, wherein the electrode is introduced into the wristband at a corresponding position, i.e. at a corresponding distance from the edge of the wristband supported on the wrist.

### SPECIFICATION OF THE CONTENT OF THE DRAWINGS

The present invention is explained in more detail below on the basis of the exemplary embodiments specified in the schematic figures of the drawings. In the drawings:

FIG. 1 shows a schematic diagram according to one embodiment of the invention;

FIG. 2 shows a schematic front view of a user interface of a device according to one embodiment of the invention;

FIG. 3 shows a schematic block diagram according to one embodiment of the invention.

The appended drawings are to provide further comprehension of the embodiments of the invention. They illustrate embodiments and serve, in conjunction with the description, to explain principles and concepts of the invention. Other embodiments and many of the mentioned advantages result with regard to the drawings. The elements in the drawings are not necessarily shown to scale in relation to one another.

In the figures of the drawings, identical, functionally identical, and identically acting elements, features, and components—unless indicated otherwise—are each provided with the same reference signs.

### DESCRIPTION OF EXEMPLARY EMBODIMENTS

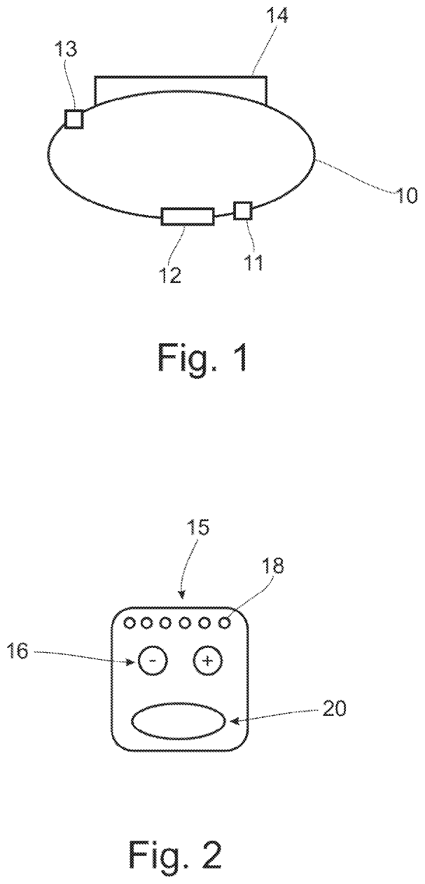

FIG. 1 shows a device 10 to be worn on the body of a person, which is formed in FIG. 1 as a wristband.

The wristband comprises an electrode 12, by means of which an electrical pulse can be transferred to the wrist or the forearm of a person.

For example, it can be provided that the electrode 12 stimulates a specific acupressure point, for example, the point P6.

For example, it can be provided that the device 10 has means, for example a camera, for this purpose, by means of which correct positioning of the device on an arm of a person can be checked. Alternatively, it is conceivable to prompt a user themselves for the correct positioning of the device 10, for example via an operating instruction.

The wristband according to FIG. 1 moreover comprises a processing unit for recognizing and evaluating sensor data. The processing unit 14 is connected via the wristband to a sensor 11 for measuring the electrodermal activity of the person.

Alternatively, it can be provided that the processing unit 14 and the sensor 11 are installed in a housing.

The device 10 moreover comprises a voltage source 13, which provides a voltage for the processing unit 14 and the sensor 11 and also the electrode 12.

FIG. 2 shows an operating interface of a user interface 15. The user interface 15 is designed as a display having a touchpad. The display comprises LEDs 18 for indicating a stimulation intensity. The touchpad comprises an operating element 20 to change between an automatic mode and a manual mode, and also operating elements 16 for manually controlling the stimulation pulses.

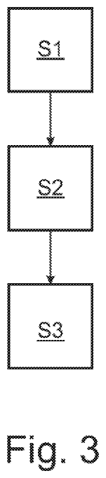

FIG. 3 shows a schematic block diagram of a method for treating kinetosis according to one embodiment of the invention. In step S1, the electrodermal activity of a person is measured by means of an EDA sensor. In step S2, it is assessed whether the person is negatively affected by kinetosis. The measured electrodermal activity is used for this purpose. In step S3, electrical pulses are generated at an electrode which is in skin contact with the person, to initiate treatment of the kinetosis on the basis of the assessment as to whether the person is negatively affected by the kinetosis. The sensor and the electrode are integrated in an object wearable on the body of the person.

### LIST OF REFERENCE SIGNS

S1-S3 method steps
10 device
11 sensor
12 electrode
13 voltage source
14 processing unit
15 user interface
16 operating elements
18 LEDs
20 operating elements

1. A method for treating kinetosis having the following steps:
measuring an electrodermal activity of a person by means of an EDA sensor;
assessing on the basis of the measured electrodermal activity whether the person is negatively affected by kinetosis;
generating electrical pulses at an electrode which is in skin contact with the person, for treating the kinetosis on the basis of the assessment as to whether the person is negatively affected by the kinetosis;
wherein the sensor and the electrode are integrated in a device wearable on the body of the person.

2. The method for treating kinetosis as claimed in claim 1, wherein an acupuncture point and/or an acupressure point is stimulated by means of the electrical pulses.

3. The method for treating kinetosis as claimed in claim 1, wherein an intensity of the electrical pulses can furthermore be input via a user input of the person into a user interface.

4. The method for treating kinetosis as claimed in claim 1, wherein furthermore the skin temperature of the person is measured by means of a thermometer and the negative effect is assessed on the basis of the skin temperature.

5. A device to be worn on the body of a person having:
a sensor for measuring the electrodermal activity of a person;
an electrode on a surface of the device;
a processing unit for assessing whether the person is negatively affected by kinetosis and for ascertaining an electrical pulse at the electrode for treating the kinetosis, and having
a voltage source for the processing unit and the electrode.

6. The device to be worn on the body of a person as claimed in claim 5, wherein the device furthermore has a thermometer for measuring the skin temperature, and the processing unit processes the skin temperature to assess whether the person is negatively affected by kinetosis.

7. The device to be worn on the body of a person as claimed in claim 5, wherein the device furthermore has a user interface, via which an intensity of the pulse is settable.

8. The device to be worn on the body of a person as claimed in claim 5, wherein the voltage source is configured to generate voltage by way of the device itself.

9. The device to be worn on the body of a person as claimed in claim 5, wherein the device is shaped in such a way that finding a position of the device on the body, in which the electrode of the device stimulates a specific acupuncture point, is ensured.
